(19) United States
(12) Patent Application Publication
(10) Pub. No.: US 2025/0072777 A1
(43) Pub. Date: Mar. 6, 2025

(54) SENSOR DEVICE AND TOILET SYSTEM

(71) Applicant: TOTO LTD., Kitakyushu-shi (JP)

(72) Inventors: Masaya Nakata, Fukuoka (JP); Motoki Kobayashi, Fukuoka (JP); Shotaro Wada, Fukuoka (JP); Yoshiyuki Kaneko, Fukuoka (JP)

(21) Appl. No.: 18/814,624

(22) Filed: Aug. 26, 2024

(30) Foreign Application Priority Data
Aug. 31, 2023 (JP) 2023-141509

(51) Int. Cl. A61B 5/026; A61B 5/00; E03D 9/00

(52) U.S. Cl. CPC A61B 5/0261; A61B 5/6887; E03D 9/00; A61B 2505/07; A61B 2560/0462

(57) ABSTRACT

A sensor device includes a laser blood flow sensor and a control device. The laser blood flow sensor is provided in a toilet room and acquires biological information that includes blood flow information of a user. The control device controls the laser blood flow sensor. The control device determines presence or absence of a human body, based on information that is acquired by the laser blood flow sensor.

### CROSS-REFERENCE TO RELATED APPLICATION(S)

This application is based upon, and claims the benefit of priority to, Japanese Patent Application No. 2023-141509, filed on Aug. 31, 2023, the entire contents of which are herein incorporated by reference.

### FIELD

A disclosed embodiment(s) relate(s) to a sensor device and a toilet system.

### BACKGROUND

A sensor device has conventionally been known that emits laser light toward a human body, receives scattered light from a body tissue, an erythrocyte(s), etc., and detects a Doppler shift of a received signal, so as to detect biological information such as pulsation information, and/or blood circulation information (for example, Japanese Patent Application Publication No. 2014-079428).

A sensor device as described above is provided in a toilet room, so that it is possible to detect biological information of a toilet user. In a case where biological information of a user is detected in a toilet room, it is desirable to determine, automatically, whether or not such a user is provided in a state where such biological information is detectable. For example, a seating sensor detects presence of absence of seating of a user, so that it is possible to determine whether or not such a user is provided in a state where biological information thereof is detectable. A seating sensor is provided on, for example, a toilet seat (for example, Japanese Patent Application Publication No. 2003-010072).

However, for example, in a case where it is determined whether or not a user is provided in a state where biological information thereof is detectable, by using a seating sensor, a plurality of sensors are needed.

### SUMMARY

According to an aspect of an embodiment, a sensor device includes a laser blood flow sensor that is provided in a toilet room and acquires biological information that includes blood flow information of a user, and a control device that controls the laser blood flow sensor, wherein the control device determines presence or absence of a human body, based on information that is acquired by the laser blood flow sensor.

### BRIEF DESCRIPTION OF DRAWING(S)

FIG. 1 is a perspective view that illustrates a toilet system according to an embodiment.

FIG. 2 is a block diagram that illustrates a communication system of a toilet system according to an embodiment.

FIG. 3 is a circuit diagram that explains an outline of a laser blood flow sensor.

FIG. 4 is a flowchart that explains a human body detection process according to an embodiment.

FIG. 5 is a flowchart that explains a biological information measurement process according to an embodiment.

FIG. 6 is a time chart in a case where a biological information detection process according to an embodiment is executed.

FIG. 7 is a flowchart that explains an adjustment process for a laser current value.

FIG. 8 is a time chart (part 1) that illustrates an example where an adjustment process for a laser current value is executed.

FIG. 9 is a time chart (part 2) that illustrates an example where an adjustment process for a laser current value is executed.

FIG. 10 is a time chart (part 3) that illustrates an example where an adjustment process for a laser current value is executed.

FIG. 11 is a time chart (part 4) that illustrates an example where an adjustment process for a laser current value is executed.

FIG. 12 is a time chart (part 5) that illustrates an example where an adjustment process for a laser current value is executed.

### DESCRIPTION OF EMBODIMENT(S)

Hereinafter, a toilet system as disclosed in the present application will be explained in detail, with reference to the accompanying drawing(s). Additionally, this invention is not limited by an embodiment(s) as illustrated below. Furthermore, the drawing(s) is/are schematic, so that it should be noted that a relationship between dimensions of respective elements, a ratio of respective elements, etc., may be different from an actual one(s). Among mutual drawings, parts with different relationships and/or ratios of mutual dimensions may also be included therein.

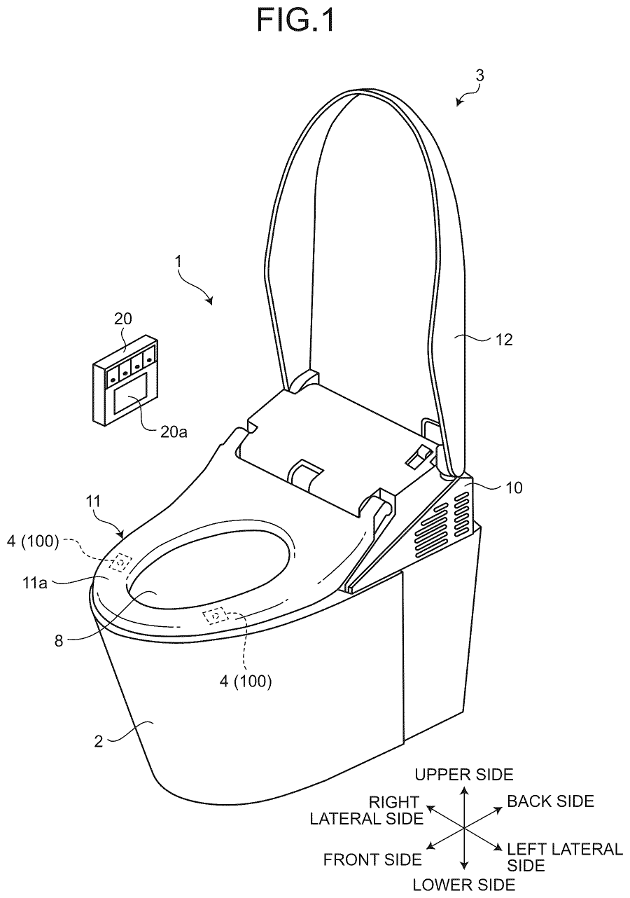

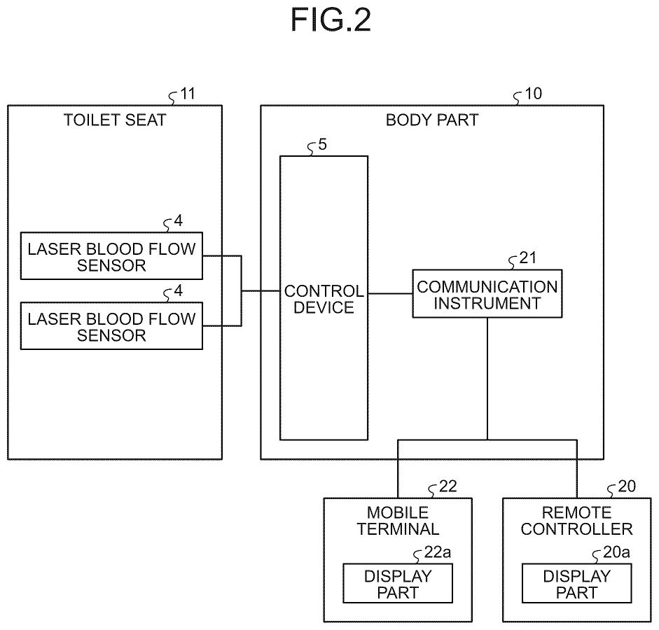

A toilet system 1 according to an embodiment will be explained with reference to FIG. 1 and FIG. 2. FIG. 1 is a perspective view that illustrates the toilet system 1 according to an embodiment. FIG. 2 is a block diagram that illustrates a communication system of the toilet system 1 according to an embodiment. Additionally, in the specification for the present application, each of an “upper side”, a “lower side”, a “front side”, a “back side”, a “left lateral side”, and a “right lateral side” is a direction that is viewed from a user that is seated on a toilet seat 11 with his/her back to a toilet lid 12 that is opened.

The toilet system 1 is provided in a toilet room. The toilet system 1 includes a toilet 2, a toilet seat device 3, a laser blood flow sensor 4, and a control device 5. The laser blood flow sensor 4 and the control device 5 are a sensor device 100 that detects biological information of a user. The laser blood flow sensor 4 may include the control device 5. The laser blood flow sensor 4 and a part of the control device 5 may be provided integrally.

The toilet 2 is a so-called sitting-style toilet. The toilet 2 has a bowl part 8 with a recess shape that is recessed downward. The toilet 2 receives waste such as urine and/or feces of a user on the bowl part 8.

The toilet seat device 3 is placed on an upper part of the toilet 2. The toilet seat device 3 may be attached to the toilet 2 integrally or may be attached to the toilet 2 to as to be attachable and detachable therefrom. The toilet seat device 3 includes a body part 10, a toilet seat 11, and a toilet lid 12.

The body part 10 is positioned at a back side of the bowl part 8 of the toilet 2 and is attached to an supper surface of the toilet 2. An opening/closing unit that controls an opening/closing action of the toilet seat 11 and the toilet lid 12, a toilet seat heating unit that controls a temperature of the toilet seat 11, a washing unit that executes washing of a private part(s) of a human body, and a deodorization unit that reduces an odor component(s) are provided in an inside of the body part 10. Furthermore, a control device that integrally controls an action of each unit and a communication instrument 21 for communicating with a remote controller 20 of a toilet room, etc., are incorporated in an inside of the body part 10.

The toilet seat 11 and the toilet lid 12 are supported so as to be rotatable with respect to the body part 10. Additionally, the toilet lid 12 is provided to the toilet seat device 3 as needed and may be omitted.

A heater wire and the laser blood flow sensor 4 are provided in an inside of the toilet seat 11. A heater wire heats a seating part 11a where a user is seated. Heating that is executed by a heater wire is controlled by a toilet seat heating unit that is provided in the body part 10.

The laser blood flow sensor 4 is an optical biological sensor. The laser blood flow sensor 4 acquires biological information of a user that is seated on the toilet seat 11. Biological information includes blood flow information. The laser blood flow sensor 4 is provided at a position where laser light is emitted toward a thigh part of a user in a case where such a user is seated on the seating part 11a. Two laser blood flow sensors 4 are provided in an inside of the toilet seat 11. The laser blood flow sensor 4 is provided so as to emit laser to both thigh parts of a user that is seated on the seating part 11a. Specifically, the two laser blood flow sensors 4 are provided so as to be symmetric with respect to a center line of the toilet seat 11 in leftward and rightward directions. Additionally, three or more laser blood flow sensors 4 may be provided thereto.

The laser blood flow sensor 4 is connected to the control device 5. Additionally, the control device 5 that is connected to the laser blood flow sensor 4 is provided separately from a control device that controls each unit. The control device 5 that is connected to the laser blood flow sensor 4 is connected to the control device 5 that controls each unit, so as to be communicable therewith. The laser blood flow sensor 4 outputs acquired biological information to the control device 5. Additionally, the control device 5 may be provided integrally with a control device that controls each unit.

The control device 5 controls the laser blood flow sensor 4 so as to measure a blood flow rate, a pulsation rate, and a pulsation variation, etc., of a user, based on biological information that is acquired from the laser blood flow sensor 4. Furthermore, the control device 5 determines presence or absence of a human body, based on information that is acquired from the laser blood flow sensor 4. Specifically, the control device 5 determines presence or absence of seating of a user. In other words, the control device 5 detects seating of a user.

The control device 5 transmits a measurement value of a blood flow rate, etc., from the communication instrument 21 to the remote controller 20 and a mobile terminal 22 of a user. Communication between the communication instrument 21 and the remote controller 20 may be wireless communication or may be wired communication. Communication between the communication instrument 21 and the mobile terminal 22 is wireless communication. Then, it is possible for a user that is seated on the toilet seat 11 to confirm a vital sign such as a blood flow rate, on a display part 20a of the remote controller 20 and/or a display part 22a of the mobile terminal 22. Additionally, a measurement value such as a blood flow rate, etc., may be transmitted to an electronic health record system for a clinical use, etc.

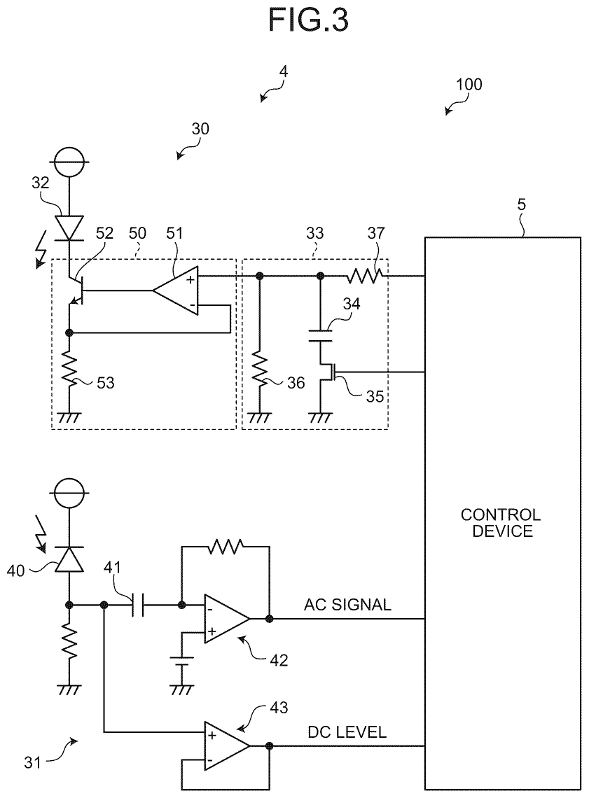

Next, a laser blood flow sensor 4 and a control device 5 will be explained with reference to FIG. 3. FIG. 3 is a circuit diagram that explains an outline of the laser blood flow sensor 4.

The laser blood flow sensor 4 is a reflection-type sensor that causes laser light that is emitted toward a thigh part of a user to reflect from a blood flow of such a user and detects such reflected light. In such an example, the laser blood flow sensor 4 uses a blood flow sensor that is capable of detecting a change of a flow rate of an erythrocyte(s) that flow(s) in a blood vessel, by using laser light. The laser blood flow sensor 4 acquires information concerning a frequency where a Doppler shift is caused by an erythrocyte(s) that flow(s) in a blood vessel.

The laser blood flow sensor 4 includes a light-emitting part 30 and a light-receiving part 31.

The light-emitting part 30 includes a laser diode 32, a stabilization circuit 33, and a constant current driving circuit 50. The laser diode 32 emits laser light. It is possible to change a light emission intensity of laser light according to a magnitude of a laser current value. Specifically, it is possible to change a light emission intensity of laser light according to a magnitude of an output voltage of the control device 5.

The stabilization circuit 33 is a circuit for stabilizing a control voltage that is supplied from the control device 5 to the constant current driving circuit 50 in a case where a laser current value is continuously supplied thereto. The stabilization circuit 33 includes a capacitor 34, a switching part 35, and resistors 36, 37.

Ground connection of the capacitor 34 is switched through the switching part 35. The switching part 35 is a switch that switches as to whether or not the capacitor 34 is connected to ground, according to a control signal that is output from the control device 5.

The constant current driving circuit 50 is a circuit that provides a constant laser current that flows through the laser diode 32. The constant current driving circuit 50 includes an operational amplifier 51, a transistor 52, and a resistor 53.

A control signal is a signal that provides a time constant for the stabilization circuit 33 as “Lo” or “Hi”. Specifically, in a case where a control signal that is output from the control device 5 is a signal that provides a time constant for the stabilization circuit 33 as “Lo”, the switching part 35 does not connect the capacitor 34 to ground. Furthermore, in a case where a control signal that is output from the control device 5 is a signal that provides a time constant for the stabilization circuit 33 as “Hi”, the switching part 35 connects the capacitor 34 to ground.

The light-receiving part 31 includes a photodiode 40, a capacitor 41, an amplification circuit 42, and a voltage follower circuit 43. The photodiode 40 receives light from an outside thereof. For example, the photodiode 40 receives reflection light of light that is emitted from the laser diode 32.

The capacitor 41 is provided so as to cut a DC component and extracts an AC component from a signal of light that is received by the photodiode 40. The capacitor 41 is provided between the amplification circuit 42 and the photodiode 40. The amplification circuit 42 amplifies an amplitude of an AC component and outputs an AC signal to the control device 5.

The voltage follower circuit 43 is connected to a path between the capacitor 41 and the photodiode 40 and outputs a DC level (a DC signal) to the control device 5. Additionally, in reflection light of laser light from a human body, an Ac component thereof is a signal that includes information of a blood flow where a Doppler shift is caused by an erythrocyte(s) and a DC component thereof is substantially equal to reflection light from a skin and/or a body tissue (where slight amount of reflection light from an erythrocyte(s) is also included).

The control device 5 is a computer. The control device 5 controls a laser current value that is supplied to the laser diode 32. The control device 5 adjusts an output voltage to the constant current driving circuit 50 through the stabilization circuit 33, so that it is possible to change a laser current value.

The control device 5 includes a first mode and a second mode as biological body detection modes. A first mode is a mode that detects presence or absence of a human body. Specifically, a first mode is a mode that determines whether or not a user has a posture for detecting biological information thereof. For example, a first mode is a mode that determines whether or not a user is seated on a toilet seat 11.

A second mode is a mode that detects biological information in a case where a human body is detected by a first mode. A second mode is a mode that is executed after a user has a posture for detecting biological information thereof, in a first mode. For example, a second mode is executed after a first mode determines that a user is seated on the toilet seat 11. Control in each mode will be described later.

An AC signal that is amplified by the amplification circuit 42 is input to the control device 5. The control device 5 AD-convers an input AC signal into a digital signal, subsequently executes a fast Fourier transform thereof, etc., and executes an frequency analysis thereof, so as to detect biological information of a user.

A DC level that is output from the voltage follower circuit 43 is input to the control device 5. The control device 5 AD-convers an input DC level, and subsequently determines presence or absence of a human body, based on an AD-converted DC level. That is, the control device 5 determines seating of a user on the toilet seat 11, based on a DC component. A determination method for seating of a user on the toilet seat 11 will be described later.

The control device 5 starts a first mode, for example, in a case where a biological body detection start button for starting biological information that is provided on a remote controller 20 is operated by a user. Additionally, the control device 5 may automatically start a first mode, independently of an operation that is executed by the remote controller 20. Specifically, the control device 5 automatically starts a first mode, for example, as a power source of a toilet seat device 3 is turned on. Furthermore, the control device 5 automatically starts a first mode, for example, as room entry of a user to a toilet booth that is provided with a toilet 2 is detected.

The control device 5 intermittently drives the laser blood flow sensor 4 in a first mode. The control device 5 repeatedly changes an output voltage between 0 V and a predetermined voltage in a first mode. A predetermined voltage is a voltage that is higher than 0 V. Furthermore, the control device 5 provides a time constant for the stabilization circuit 33 as “Lo” in a first mode. That is, in a first mode, a state where the capacitor 34 is not connected to ground is provided by the switching part 35.

In a first mode, the capacitor 34 is not connected to ground, so that laser light is emitted from the laser diode 32, depending on a pulse waveform of an output voltage that is output from the control device 5. Hence, in a first mode, the laser blood flow sensor 4 is driven intermittently.

The control device 5 continuously drives the laser blood flow sensor 4 in a second mode. The control device 5 provides an output voltage as a predetermined voltage in a second mode. Furthermore, the control device 5 provides a time constant for the stabilization circuit 33 as “Hi” in a second mode. That is, in a second mode, a state where the capacitor 34 is connected to ground is provided by the switching part 35. Additionally, continuous driving includes pulse driving (intermittent driving) of laser light that is capable of detecting biological information of a user, for a long period of time. That is, continuous driving includes driving where a period of time when an output voltage is provided as a predetermined voltage is longer than intermittent driving in a first mode.

In a second mode, the capacitor 34 is connected to ground, so that a laser current value that is supplied to the laser diode 32 is stabilized and output of laser light that is emitted from the laser diode 32 is stabilized, even in a case where a tiny variation is caused in an output voltage that is output from the control device 5.

Next, a biological information detection process according to an embodiment will be explained. A biological information detection process includes a human body detection process and a biological information measurement process.

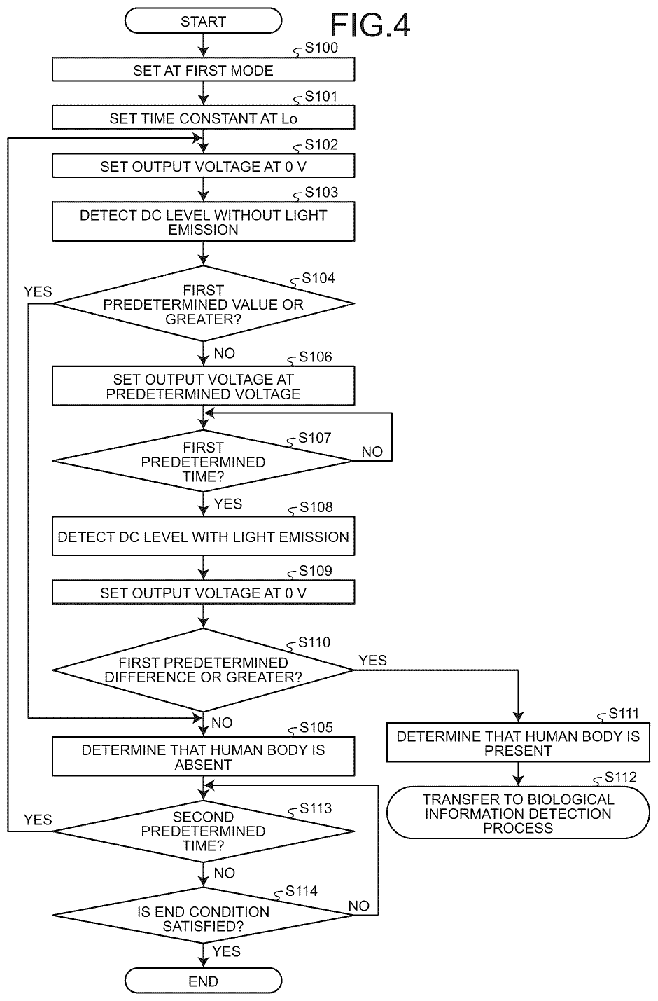

First, a human body detection process will be explained with reference to a flowchart in FIG. 4. FIG. 4 is a flowchart that explains a human body detection process according to an embodiment. A human body detection process is started by, for example, operating a biological information detection start button of a remote controller 20. Furthermore, a human body detection process is started in a case where it is determined that a human body is absent, in a biological information measurement process, as described later in detail. Additionally, a human body detection process may automatically be started, independently of an operation of a biological information detection start button of the remote controller 20. A human body detection process may be stated as a power source of a toilet seat device 3 is turned on, and be executed constantly in a case where such a power source of the toilet seat device 3 is turned on. Furthermore, a human body detection process may automatically be started as a user in a toilet booth is detected.

A control device 5 sets a biological body detection mode at a first mode (S100). Then, the control device 5 sets a time constant for a stabilization circuit 33 at “Lo” (S101). That is, the control device 5 provides a state where a capacitor 34 of the stabilization circuit 33 is not connected to ground.

Then, the control device 5 sets an output voltage at 0 V (S102). The capacitor 34 of the stabilization circuit 33 is not connected to ground and an output voltage is 0 V, so that a laser current value is zero and laser light is not emitted from a laser diode 32.

Then, the control device 5 detects a DC level (S103). The control device 5 detects a DC level in a state where the laser diode 32 does not emit laser light (that will be referred to as a “DC level without light emission” below). A DC level without light emission means a brightness of an ambient environment with light that is received by a photodiode 40. That is, an outside light level is provided.

Then, the control device 5 determines whether or not a DC level without light emission is a first predetermined value or greater (S104). A first predetermined value is a preliminarily set value and is a maximum value that is allowable for outside light. The control device 5 determines that a human body is absent (S105) in a case where a DC level without light emission is a first predetermined value or greater (S104: Yes). That is, the control device 5 determines that a user is not seated on a toilet seat 11 in a case where a DC level without light emission is a first predetermined value or greater.

The control device 5 sets an output voltage at a predetermined voltage (S106) in a case where a DC level without light emission is less than a first predetermined value (S104: No). Thereby, a laser current value is increased depending on a predetermined voltage, so that laser light is emitted from the laser diode 32.

Then, the control device 5 determines whether or not a first elapsed time after an output voltage is provided as a predetermined voltage is a first predetermined time (S107). A first predetermined time is a preliminarily set time and a very short period of time. A first predetermined time is, for example, 10 μs. The control device 5 repeats a process at step S107 and waits in a case where a first elapsed time is not a first predetermined time (S107: No).

The control device 5 detects a DC level (S108) in a case where a first elapsed time is a first predetermined time (S107: Yes). The control device 5 detects a DC level in a state where the laser diode 32 emits laser light (that will be referred to as a “DC level with light emission” below).

Then, the control device 5 sets an output voltage at 0 V (S109). Thereby, emission of laser light from the laser diode 32 is stopped.

Then, the control device 5 determines whether or not a difference between a DC level with light emission and a DC level without light emission is a first predetermined difference or greater (S110). A first predetermined difference is a preliminarily set value. In a case where a human body is present, that is, a case where a user is seated on the toilet seat 11, light that is reflected from a user is present, so that a DC level with light emission is increased. Hence, a DC level with light emission in a case where a user is seated on the toilet seat 11 is greater than a DC level with light emission in a case where such a user is not seated on the toilet seat 11. A first predetermined difference is set at a value that is capable of determining seating of a user on the toilet seat 11. For example, a first predetermined difference is set, depending on a result of an experiment, a simulation, etc.

The control device 5 determines that a human body is present (S111) in a case where a difference between a DC level with light emission and a DC level without light emission is a first predetermined difference or greater (S110: Yes). That is, the control device 5 determines that a user is seated on the toilet seat 11. The control device 5 transfers to a biological information detection process as described later (S112) in a case where it is determined that a user is seated on the toilet seat 11.

The control device 5 determines that a human body is absent (S105) in a case where a difference between a DC level with light emission and a DC level without light emission is less than a first predetermined difference (S110: No). That is, the control device 5 determines that a user is not seated on the toilet seat 11.

Then, the control device 5 determines whether or not a second elapsed time after an output voltage is provided as 0 V is a second predetermined time (S113). That is, the control device 5 determines whether or not a second elapsed time after emission of laser light from the laser diode 32 is stopped is a second predetermined time. A second predetermined time is a preliminarily set time and is a period of time that is longer than a first predetermined time. For example, a second predetermined time is 10 ms.

The control device 5 returns to step S102 and repeats a process as described above in a case where a second elapsed time is a second predetermined time (S113: Yes).

Thus, the control device 5 in a first mode repeatedly executes emission of laser light that is executed by the laser diode 32 at an interval(s) of a second predetermined time, intermittently drives a laser blood flow sensor 4, and determines presence or absence of a human body based on a DC component.

In a first mode, the capacitor 34 of the stabilization circuit 33 is not connected to ground, so that it is possible for the laser blood flow sensor 4 to derive the laser diode 32 intermittently, instantly in response to a timing when an output voltage is switched into 0 V or a predetermined voltage.

The control device 5 determines whether or not an end condition for a biological information detection process is satisfied (S114) in a case where a second elapsed time is not a second predetermined time (S113: No). For example, the control device 5 determines that an end condition for a biological information detection process is satisfied in a case where a biological information detection end button of the remote controller 20 is operated. The control device 5 determines that an end condition for a biological information detection process is not satisfied in a case where a biological information detection end button of the remote controller 20 is not operated.

The control device 5 ends a current process in a case where an end condition for a biological information detection process is satisfied (S114: Yes).

The control device 5 returns to step S113 and repeats a process as described above in a case where an end condition for a biological information detection process is not satisfied (S114: No).

Additionally, the control device 5 may determine that an end condition for a biological information detection process is satisfied in a case where a measurement end time has passed after a human body detection process is started. A measurement end time is a preliminarily set time and is, for example, a common period of time of use of a toilet that is executed by a user. Additionally, as described above, a human body detection process may be executed constantly, for example, in a case where a power source of the toilet seat device 3 is turned on, and be continued independently of passage of a measurement end time.

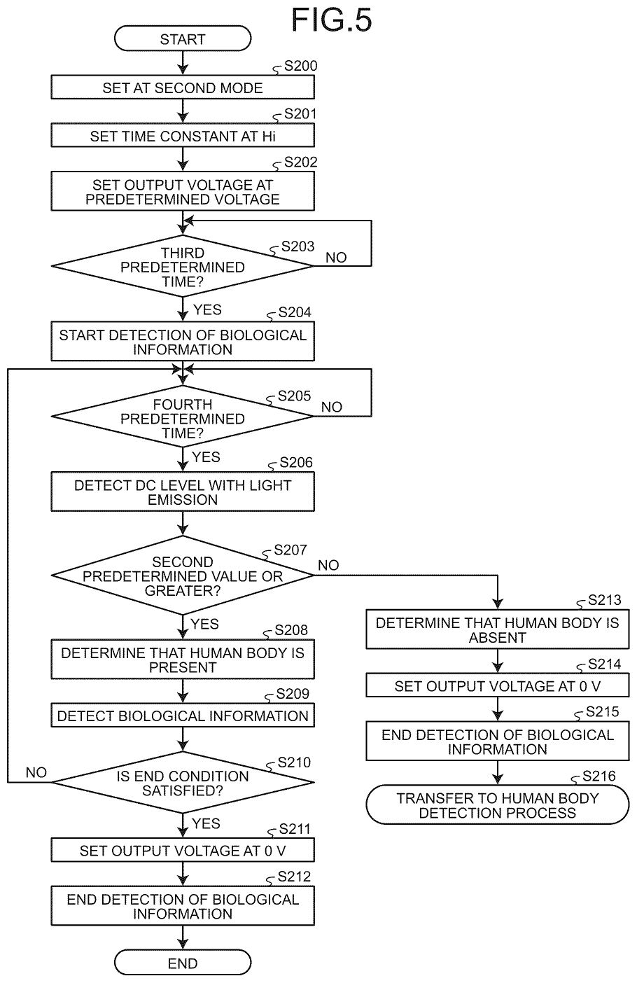

Next, a biological information measurement process will be explained with reference to FIG. 5. FIG. 5 is a flowchart that explains a biological information measurement process according to an embodiment. A biological information measurement process is started in a case where it is determined that a human body is present, in a human body detection process (S110).

A control device 5 sets a biological body detection mode at a second mode (S200). Then, the control device 5 sets a time constant for a stabilization circuit 33 at “Hi” (S201). That is, the control device 5 provides a state where a capacitor 34 of the stabilization circuit 33 is connected to ground.

Then, the control device 5 sets an output voltage at a predetermined voltage (S202). Thereby, laser light is emitted from a laser diode 32. Additionally, in a second mode, a time constant for the stabilization circuit 33 is set at “Hi”, so that the laser diode 32 continuously emits laser light. That is, in a second mode, a laser blood flow sensor 4 is driven continuously.

Then, the control device 5 determines whether or not a third elapsed time after an output voltage is provided as a predetermined voltage is a third predetermined time (S203). A third predetermined time is a preliminarily set time and a period of time when the capacitor 34 is charged. That is, a third predetermined time is a period of time until a laser current value of laser light that is emitted from the laser diode 32 is stabilized by the stabilization circuit 33. The control device 5 repeats a process at step S203 and waits in a case where a third elapsed time is not a third predetermined time (S203: No).

The control device 5 starts detection of biological information of a user based on an AC signal (S204) in a case where a third elapsed time is a third predetermined time (S203: Yes).

Then, the control device 5 determines whether or not a fourth elapsed time is a fourth predetermined time (S205). A fourth predetermined time is a sampling period for detecting biological information of a user. That is, the control device 5 determines whether or not a sampling period has passed. For example, the control device 5 determines whether or not a fourth elapsed time after detection of biological information of a user is started is a fourth predetermined time.

The control device 5 repeats a process at step S205 and waits in a case where a fourth elapsed time is not a fourth predetermined time (S205: No). The control device 5 detects a DC level (S206) in a case where a fourth elapsed time is a fourth predetermined time (S205: Yes). The control device 5 detects a DC level with light emission.

Then, the control device 5 determines whether or not a DC level with light emission is a second predetermined value or greater (S207). A second predetermined value is a preliminarily set value and is a DC level that is detected in a case where a user is seated on a toilet seat 11. For example, a second predetermined value is set, depending on a result of an experiment, a simulation, etc.

The control device 5 determines that a human body is present (S208) in a case where a DC level with light emission is a second predetermined value or greater (S207: Yes). That is, the control device 5 determines that a state where a user is seated on the toilet seat 11 is continued.

Then, the control device 5 detects biological information of a user, based on an AC signal that is acquired during a fourth predetermined time (S209). For example, the control device 5 calculates blood flow information and/or pulsation information of a user.

Then, the control device 5 determines whether or not an end condition for a biological information detection process is satisfied (S210). The control device 5 returns to step S205 and repeats a process as described above in a case where an end condition for a biological information detection process is not satisfied (S210: No). That is, the control device 5 repeats detection of biological information of a user that is based on an AC signal and determination of presence or absence of a human body.

The control device 5 sets an output voltage at 0 V (S211) in a case where an end condition for a biological information detection process is satisfied (S210: Yes). Thereby, emission of laser light from the laser diode 32 is stopped.

Then, the control device 5 ends detection of biological information of a user that is based on an AC signal (S212), so as to end a biological information detection process.

The control device 5 determines that a human body is absent (S213) in a case where a DC level with light emission is less than a second predetermined value (S207: No). That is, the control device 5 determines that a user is not seated on the toilet seat 11.

Then, the control device 5 sets an output voltage at 0 V (S214). Thereby, emission of laser light from the laser diode 32 is stopped.

Then, the control device 5 ends detection of biological information of a user based on an AC signal (S215) and transfers to a human body detection process (S216).

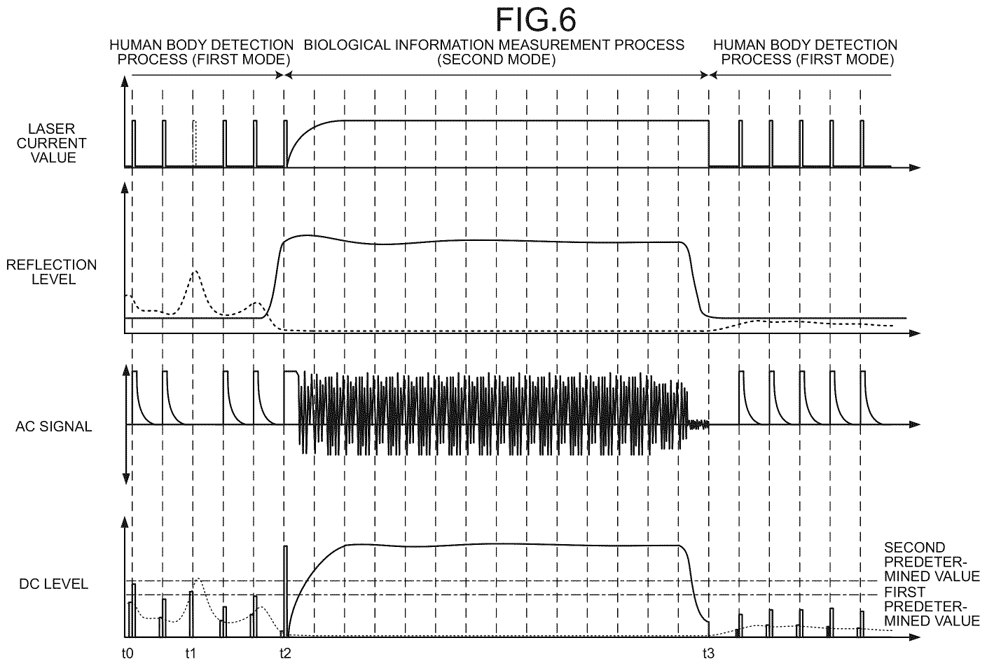

Next, an example where a biological information detection process according to an embodiment is executed will be explained with reference to FIG. 6. FIG. 6 is a time chart in a case where a biological information detection process according to an embodiment is executed. Herein, it is assumed that a user operates a biological information detection start button of a remote controller 20 so as to start a human body detection process.

FIG. 6 illustrates a laser current value of a laser diode 32, a reflection level, an AC signal, and a DC level from an upper section thereof. Additionally, a reflection level indicates an amount of received light in a case where the laser diode 32 emits light. A reflection level does not include an amount of received light for outside light. That is, a reflection level does not include outside light, and hence, is not a received light level of a photodiode 40. Furthermore, an example of a states of a reflection level and a DC level for outside light is indicated by a broken line.

In a human body detection process, a biological body detection mode is set at a first mode, so that a laser blood flow sensor 4 is driven intermittently so as to emit laser light intermittently.

At time to, a DC level without light emission is detected in a state where the laser diode 32 does not emit light. Herein, a detected DC level without light emission is less than a first predetermined value, so that laser light is subsequently emitted from the laser diode 32 and a DC level with light emission is detected.

Herein, it is determined that a DC level with light emission is small and a difference between such a DC level with light emission and a DC level without light emission is less than a first predetermined difference, so that a human body is absent, that is, a user is not seated on the toilet seat 11. Hence, a human body detection process is continued. Additionally, a reflection level at a time when a user is not seated on the toilet seat 11 is, for example, a level that is dependent on an internal reflection of a case of the laser blood flow sensor 4 in a case where laser light is emitted, and is a constant level that is sufficiently less than reflection at a time when a user is seated on the toilet seat 11.

Subsequently, outside light is increased, so that a DC level without light emission that is detected at a time t1 is a first predetermined value or greater (where it is assumed that outside light is so changed at a time t1). Hence, laser light is not emitted from the laser diode 32 herein.

Subsequently, a detected DC level with light emission is increased and a difference between a DC level with light emission and a DC level without light emission is a first predetermined difference or greater. Then, at a time t2, it is determined that a human body is present, that is, a user is seated on the toilet seat 11. Thereby, a process transfers to a biological information measurement process.

In a biological information measurement process, a biological body detection mode is set at a second mode, so that the laser blood flow sensor 4 is driven continuously so as to emit laser light continuously. A reflection level includes reflection light from a human body, and hence, is increased. Furthermore, a DC level with light emission is increased. Moreover, an AC signal has a waveform of a light beat signal that is scattered from an erythrocyte(s) of a human body. In a biological information measurement process, biological information of a user is detected based on an AC signal.

subsequently, as a user is left from the toilet seat 11, reflection light from a human body is absent, so that a DC level with light emission is decreased. Then, at a time t3, in a case where a DC level with light emission is less than a second predetermined value, it is determined that a human body is absent, that is, a user is left from the toilet seat 11. Thereby, a process transfers to a human body detection process. In a human body detection process, a biological body detection mode is subsequently set at a first mode, so that the laser blood flow sensor 4 is driven intermittently so as to emit laser light intermittently.

Furthermore, a control device 5 is capable of adjusting a laser current value of the laser diode 32. Specifically, the control device 5 adjusts a predetermined voltage for an output voltage so as to adjust a laser current value of the laser diode 32.

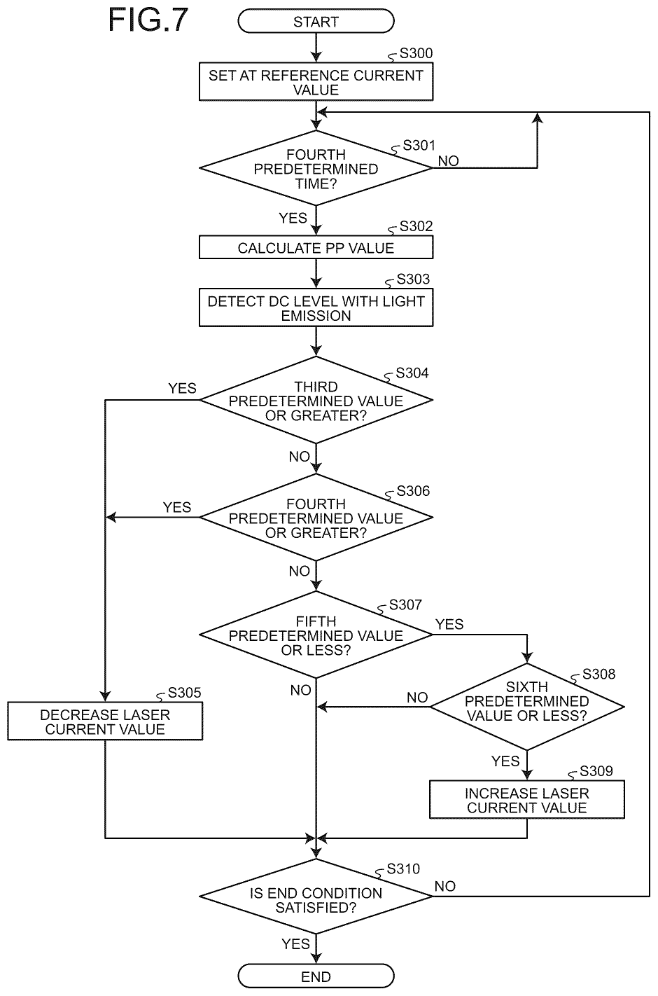

Next, an adjustment process for a laser current value will be explained with reference to FIG. 7. FIG. 7 is a flowchart that explains an adjustment process for a laser current value. An adjustment process for a laser current value is executed together with a biological information measurement process in a case where such a biological information measurement process is executed.

A control device 5 sets a laser current value at a preliminarily set reference current value (S300). Specifically, the control device 5 set a predetermined voltage for an output voltage at a reference voltage in such a manner that a laser current value is a reference current value.

The control device 5 determines whether or not a fourth elapsed time is a fourth predetermined time, in a biological information measurement process (S301). That is, the control device 5 determines whether or not a sampling period to detect biological information of a user has passed. In a case where a fourth elapsed time is not a fourth predetermined time, a process at step S301 is predetermined and waiting is executed.

The control device 5 calculates a PP value (a peak-to-peak value) of an AC signal that is detected during a fourth elapsed time (S302) in a case where such a fourth elapsed time is a fourth predetermined time (S301: Yes).

Then, the control device 5 detects a DC level with light emission (S303). The control device 5 detects a DC level with light emission during a fourth elapsed time. Then, the control device 5 determines whether or not a DC level with light emission is a third predetermined value or greater (S304). A third predetermined value is a preliminarily set value. A third predetermined value is a maximum value that is allowable for a DC level with light emission. A third predetermined value is a value for reducing or preventing saturation of a laser blood flow sensor 4.

The control device 5 decreases a laser current value (S305) in a case where a DC level with light emission is a third predetermined value or greater (S304: Yes). Specifically, the control device 5 decreases a predetermined voltage for an output voltage in such a manner that a laser current value is decreased at a first predetermined rate. A first predetermined rate is a preliminarily set rate, and is, for example, 5%. The control device 5 transfers a process to step S310 after a laser current value is decreased.

The control device 5 determines whether or not a PP value of an AC signal is a fourth predetermined value or greater (S306) in a case where a DC level with light emission is less than a third predetermined value (S304: No). A fourth predetermined value is a preliminarily set value. A fourth predetermined value is a maximum value that is allowable for an amplitude of an AC signal.

The control device 5 decreases a laser current value (S305) in a case where a PP value of an AC signal is a fourth predetermined value or greater (S306: Yes).

The control device 5 determines whether or not a PP value of an AC signal is a fifth predetermined value or less (S307) in a case where such a PP value of an AC signal is less than a fourth predetermined value (S306: No). A fifth predetermined value is a preliminarily set value. A fifth predetermined value is less than a fourth predetermined value. A fifth predetermined value is a minimum value that is allowable for an amplitude of an AC signal.

The control device 5 transfers a process to step S310 in a case where a PP value of an AC signal is less than a fifth predetermined value (S307: No).

The control device 5 determines whether or not a DC level with light emission is a sixth predetermined value or less (S308) in a case where a PP value of an AC signal is a fifth predetermined value or less (S307: Yes). A sixth predetermined value is a preliminarily set value. A sixth predetermined value is less than a third predetermined value. A sixth predetermined value is, for example, a value that is 95% of a third predetermined value. Herein, the control device 5 determines whether or not a DC level with light emission has a margin of an increase for a third predetermined value.

The control device 5 transfers a process to step S310 in a case where a DC level with light emission is greater than a sixth predetermined value (S308: No).

The control device 5 increases a laser current value (S309) in a case where a DC level with light emission is a sixth predetermined value or less (S308: Yes). Specifically, the control device 5 increases a predetermined voltage for an output voltage in such a manner that a laser current value is increased at a second predetermined rate. A second predetermined rate is a preliminarily set rate, and is, for example, 5%. The control device 5 transfers to step S310 after a laser current value is increased.

The control device 5 determines whether or not an end condition for a biological information detection process is satisfied (S310). The control device 5 returns to step S301 and repeats a process as described above in a case where an end condition for a biological information detection process is not satisfied (S310: No).

The control device 5 ends a current process in a case where an end condition for a biological information detection process is satisfied (S310: Yes).

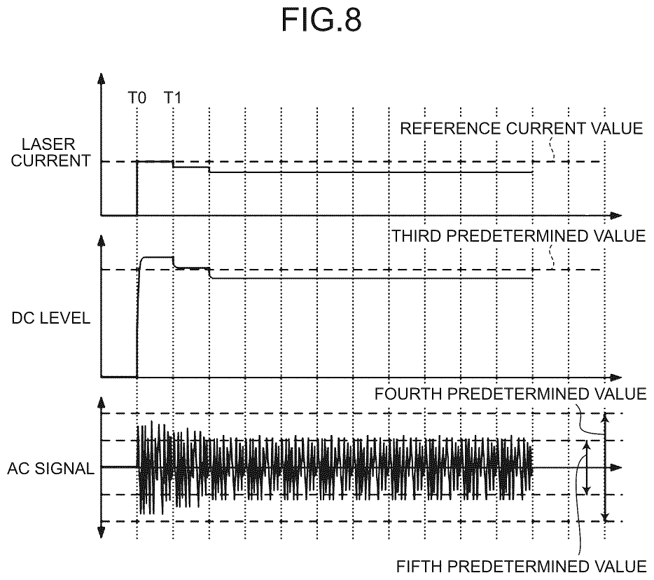

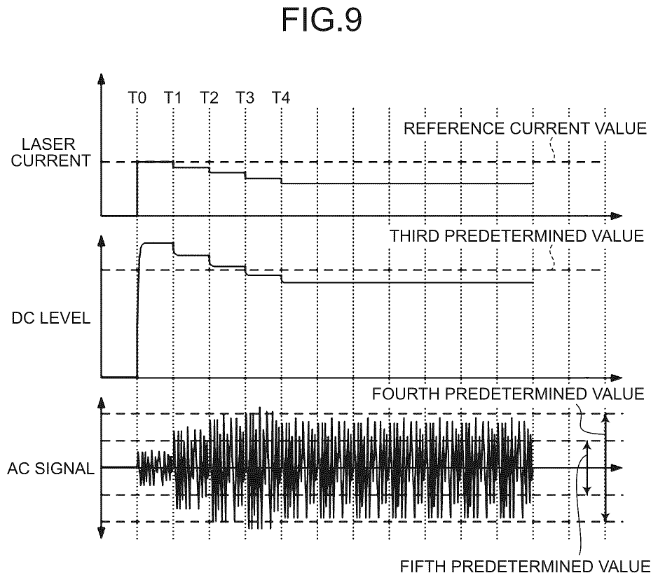

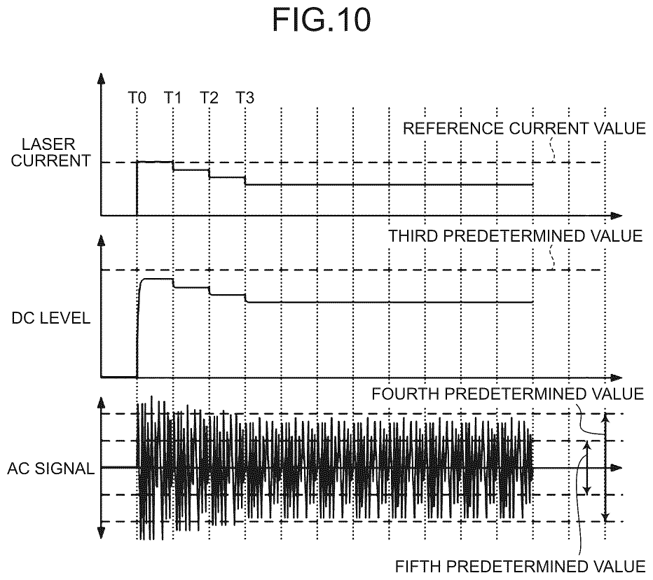

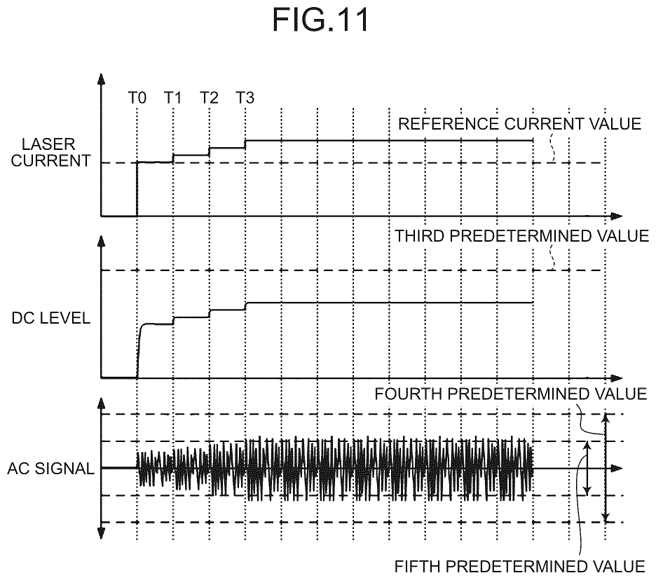

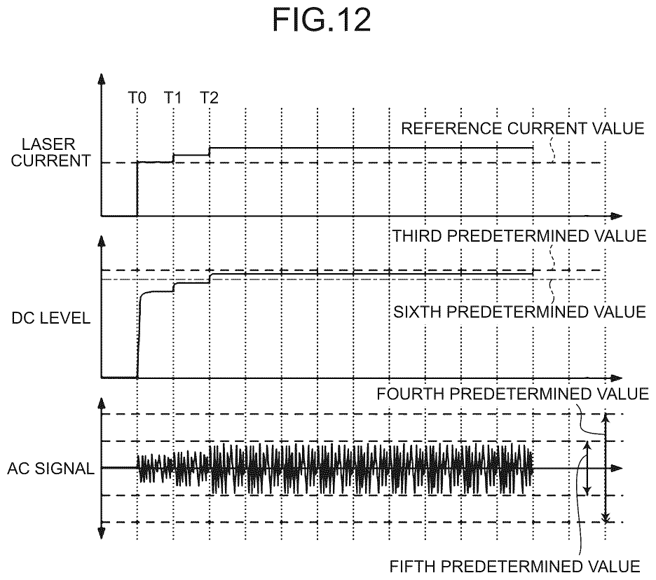

Next, a laser current value, a DC level with light emission, and an AC signal in a case where an adjustment process for a laser current value is executed will be explained with reference to FIG. 8 to FIG. 12. FIG. 8 is a time chart (part 1) that illustrates an example where an adjustment process for a laser current value is executed. FIG. 9 is a time chart (part 2) that illustrates an example where an adjustment process for a laser current value is executed. FIG. 10 is a time chart (part 3) that illustrates an example where an adjustment process for a laser current value is executed. FIG. 11 is a time chart (part 4) that illustrates an example where an adjustment process for a laser current value is executed. FIG. 12 is a time chart (part 5) that illustrates an example where an adjustment process for a laser current value is executed. Additionally, in FIG. 8 to FIG. 12, a longitudinal broken line corresponds to a sampling period, that is, passage of a fourth predetermined time.

As illustrated in FIG. 8, after a laser current value is set at a reference current value at a time TO, a DC level with light emission is a third predetermined value or greater at a time T1. Hence, a laser current value is decreased. A DC level with light emission is also a third predetermined value or greater at a time T2. Hence, a laser current value is decreased. Subsequently, a DC level with light emission is less than a third predetermined value, so that a laser current value is maintained. Additionally, in FIG. 8, a PP value of an AC signal is less than a fourth predetermined value and is a fifth predetermined value or greater.

Thus, in a case where a DC level with light emission is a third predetermined value or greater, a laser current value is decreased so as to reduce or prevent saturation of a laser blood flow sensor 4. Additionally, saturation means that a DC current of a photodiode 40 is increased, a voltage of a resistor in a lower section approaches a power source voltage, and such a voltage is not changed.

As illustrated in FIG. 9, after laser current value is set at a reference current value at a time TO, a DC level with light emission is a third predetermined value or greater at a time T1. Hence, a laser current value is decreased. A DC level with light emission is also a third predetermined value or greater at a time T2. Hence, a laser current value is decreased. A DC level with light emission is also a third predetermined value or greater at a time T3. Hence, a laser current value is decreased. Although a DC level with light emission is less than a third predetermined value at a time T4, a PP value of an AC signal from a time T3 to a time T4 is a fourth predetermined value or greater. There is a concern that this exceeds an output range of an amplification circuit 42 and an input range (an accuracy assurance range) of A/D conversion of a control device 5. Hence, a laser current value is also decreased at a time T4. Subsequently, a DC level with light emission is less than a third predetermined value and a PP value of an AC signal is less than a fourth predetermined value, so that a laser current value is maintained. Additionally, in FIG. 9, a PP value of an AC signal is greater than a fifth predetermined value.

As illustrated in FIG. 10, after a laser current value is set at a reference current value at a time TO, although a DC level with light emission is less than a third predetermined value at a time T1, a PP value of an AC signal from a time T0 to T1 is a fourth predetermined value or greater. Hence, a laser current value is decreased. Although a DC level with light emission is also less than a third predetermined value at a time T2, a PP value of an AC signal from a time T1 to T2 is a fourth predetermined value or greater. Hence, a laser current value is decreased. Although a DC level with light emission is also less than a third predetermined value at a time T3, a PP value of an AC signal from a time T2 to T3 is a fourth predetermined value or greater. Hence, a laser current value is decreased. Subsequently, a PP value of an AC signal is less than a third predetermined value, so that a laser current value is maintained. Additionally, in FIG. 10, a laser current value is decreased, so that a DC level with light emission is also decreased. Furthermore, in FIG. 10, a PP value of an AC signal is greater than a fifth predetermined value.

As illustrated in FIG. 11, after a laser current value is set at a reference current value at a time TO, although a DC level with light emission is less than a third predetermined value at a time T1, a PP value of an AC signal from a time T0 to T1 is a fifth predetermined value or less. Hence, a laser current value is increased. Additionally, a DC level with light emission is sufficiently less than a third predetermined value and a sixth predetermined value or less herein. Although a DC level with light emission is also less than a third predetermined value at a time T2, a PP value of an AC signal from a time T1 to T2 is a fifth predetermined value or less. Hence, a laser current value is increased. Although a DC level with light emission is also less than a third predetermined value at a time T3, a PP value of an AC signal from a time T2 to T3 is a fifth predetermined value or less. Hence, a laser current value is increased. Subsequently, a PP value of an AC signal is greater than a fifth predetermined value, so that a laser current value is maintained. Additionally, in FIG. 11, a laser current value is increased, so that a DC level with light emission is also increased but is a sixth predetermined value or less.

As illustrated in FIG. 12, after a laser current value is set at a reference current value at a time TO, although a DC level with light emission is less than a third predetermined value at a time T1, a PP value of an AC signal from a time T0 to T1 is a fifth predetermined value or less. Hence, a laser current value is increased. Although a DC level with light emission is less than a third predetermined value at a time T2, a PP value of an AC signal from a time T1 to T2 is a fifth predetermined value or less. However, a DC level with light emission is greater than a sixth predetermined value. Hence, as a laser current value is increased, a DC level with light emission may be a third predetermined value or greater. Therefore, a laser current value is maintained.

Thus, a laser current value is adjusted. A DC level of light that is received by a photodiode 40, etc., may be changed by a feature of a user (for example, a color of skin of a user and/or an amount of body hair of a user). Hence, the control device 5 adjusts a laser current value, so that it is possible to detect biological information of a user even in a case where a feature of such a user is different.

A sensor device 100 includes the laser blood flow sensor 4 and the control device 5. The laser blood flow sensor 4 is provided in a toilet room and acquires biological information that includes blood flow information of a user. The control device 5 controls the laser blood flow sensor 4. The control device 5 determines presence or absence of a human body, based on information that is acquired by the laser blood flow sensor 4.

Thereby, it is possible for the sensor device 100 to determine presence or absence of a human body and detect biological information of a user, by the laser blood flow sensor 4. For example, it is possible for the sensor device 100 to detect seating of a user without using a seating sensor. Furthermore, for example, it is possible to eliminate a space for providing a seating sensor in a toilet seat 11 and it is possible to provide a plurality of laser blood flow sensors 4 in the toilet seat 11. Hence, it is possible for the sensor device 100 to detect biological information of a user by using, for example, the plurality of laser blood flow sensors 4, so that it is possible to improve detection accuracy of such biological information.

Furthermore, the control device 5 includes a first mode that determines presence or absence of a human body and a second mode that detects biological information in a case where it is determined that such a human body is present, in such a first mode.

Thereby, it is possible for the sensor device 100 to detect biological information of a user in a second mode after it is determined that a human body is present in a first mode, and it is possible to detect presence or absence of such a human body and detect such biological information of a user, by using the laser blood flow sensor 4.

Furthermore, the control device 5 intermittently drives the laser blood flow sensor 4 in a first mode and continuously drives the laser blood flow sensor 4 in a second mode.

Thereby, the sensor device 100 intermittently drives the laser blood flow sensor 4 in a first mode, so that it is possible to reduce a strain that is applied to an eye(s) of a user in a case where laser light from the laser blood flow sensor 4 is viewed thereby, for example, before a user seats thereon. Furthermore, a user is seated thereon in a case where laser light is continuously emitted from the laser blood flow sensor 4, so that it is possible for the sensor device 100 to reduce or prevent emitting of laser light to an eye(s) of a user. That is, it is possible for the sensor device 100 to improve safety of a user. Furthermore, it is possible for the sensor device 100 to decrease a period of time when laser light is emitted continuously while detection of presence or absence of a human body and detection of biological information of a user are executed by using the laser blood flow sensor 4, so that it is possible to extend a life of the laser blood flow sensor 4.

Furthermore, the control device 5 determines presence or absence of a human body based on a DC component of light that is received by the laser blood flow sensor 4 in a first mode, and detects biological information based on an AC component of light that is received by the laser blood flow sensor 4 in a second mode.

Thereby, it is possible for the sensor device 100 to determine presence or absence of a human body in a case where presence or absence of a human body is determined in a first mode, for a period of time that is less than that of a case where such presence or absence of a human body is determined by using an AC component.

An aspect of an embodiment aims to provide a sensor device and a toilet system that reduce a number of a sensor(s) to be used and detect biological information of a user.

A sensor device according to an aspect of an embodiment includes a laser blood flow sensor and a control device. The laser blood flow sensor is provided in a toilet room and acquires biological information that includes blood flow information of a user. The control device controls the laser blood flow sensor. The control device determines presence or absence of a human body, based on information that is acquired by the laser blood flow sensor.

Thereby, it is possible for a sensor device to determine presence or absence of a human body and detect biological information of a user, by a laser blood flow sensor. For example, it is possible for a sensor device to detect seating of a user without using a seating sensor. Furthermore, for example, it is possible to eliminate a space where a seating sensor is provided in a toilet seat, and it is possible to provide a plurality of laser blood flow sensors on such a toilet seat. Hence, it is possible for a sensor device to detect biological information of a user by using, for example, a plurality of laser blood flow sensors, so that it is possible to improve detection accuracy for such biological information.

Furthermore, the control device includes a first mode that determines presence or absence of a human body, and a second mode that detects the biological information, in a case where the first mode determines that a human body is present.

Thereby, it is possible for a sensor device to detect biological information of a user in a second mode after a first mode determines that a human body is present, and it is possible to detect presence or absence of such a human body and detect such biological information of a user by using a laser blood flow sensor.

Furthermore, the control device intermittently drives the laser blood flow sensor, in the first mode, and continuously drives the laser blood flow sensor, in the second mode.

Thereby, a sensor device intermittently drives a laser blood flow sensor in a first mode, so that it is possible to reduce a strain that is applied to an eye(s) of a user in a case where such a user sees laser light from a laser blood flow sensor, for example, before seating thereof. Furthermore, in a case where laser light is continuously emitted from a laser blood flow sensor, a user is seated, so that it is possible for a sensor device to reduce or prevent emitting of such laser light to an eye(s) of such a user. That is, it is possible for a sensor device to improve safety of a user. Furthermore, it is possible for a sensor device to decrease a period of time when laser light is continuously emitted while detection of presence or absence of a human body and detection of biological information of a user are executed, by using a laser blood flow senor, so that it is possible to extend a life of such a laser blood flow sensor.

Furthermore, the control device determines presence or absence of a human body based on a DC component of light that is received by the laser blood flow sensor, in the first mode, and detects the biological information based on an AC component of light that is received by the laser blood flow sensor, in the second mode.

Thereby, it is possible for a sensor device to determine present or absence of a human body in a case where a first mode determines such presence or absence of a human body, for a period of time that is less than that in a case where such presence or absence of a human body is determined by using an AC component.

According to an aspect of an embodiment, it is possible for a sensor device and a toilet system to reduce a number of a sensor(s) to be used and detect biological information of a user accurately.

It is possible for a person(s) skilled in the art to readily derive an additional effect(s) and/or variation(s). Hence, a broader aspect(s) of the present invention is/are not limited to a specific detail(s) and a representative embodiment(s) as illustrated and described above. Therefore, various modifications are possible without departing from the spirit or scope of a general inventive concept that is defined by the appended claim(s) and an equivalent(s) thereof.

<Appendix> (1) A sensor device, including:
a laser blood flow sensor that is provided in a toilet room and acquires biological information that includes blood flow information of a user; and
a control device that controls the laser blood flow sensor, wherein
the control device determines presence or absence of a human body, based on information that is acquired by the laser blood flow sensor.
(2) The sensor device according to (1), wherein
the control device includes:
   a first mode that determines presence or absence of a human body; and
   a second mode that detects the biological information, in a case where the first mode determines that a human body is present.
(3) The sensor device according to (2), wherein
the control device:
   intermittently drives the laser blood flow sensor, in the first mode; and
   continuously drives the laser blood flow sensor, in the second mode.
(4) The sensor device according to (2) or (3), wherein
the control device:
   determines presence or absence of a human body based on a DC component of light that is received by the laser blood flow sensor, in the first mode; and
   detects the biological information based on an AC component of light that is received by the laser blood flow sensor, in the second mode.
(5) A toilet system, including
the sensor device according to any one of (1) to (4).

What is claimed is:

1. A sensor device, comprising:
a laser blood flow sensor that is provided in a toilet room and acquires biological information that includes blood flow information of a user; and
a control device that controls the laser blood flow sensor, wherein
the control device determines presence or absence of a human body, based on information that is acquired by the laser blood flow sensor.

2. The sensor device according to claim 1, wherein
the control device includes:
a first mode that determines presence or absence of a human body; and
a second mode that detects the biological information, in a case where the first mode determines that a human body is present.

3. The sensor device according to claim 2, wherein
the control device:
intermittently drives the laser blood flow sensor, in the first mode; and
continuously drives the laser blood flow sensor, in the second mode.

4. The sensor device according to claim 2, wherein
the control device:
determines presence or absence of a human body based on a DC component of light that is received by the laser blood flow sensor, in the first mode; and
detects the biological information based on an AC component of light that is received by the laser blood flow sensor, in the second mode.

5. A toilet system, comprising:
a toilet;
a toilet seat device that is placed on an upper part of the toilet; and
the sensor device according to claim 1,
wherein the sensor device is provided in the toilet seat device.
